(19) United States
(12) Patent Application Publication
(10) Pub. No.: US 2012/0232641 A1
(43) Pub. Date: Sep. 13, 2012

(54) PERCUTANEOUS ENDOPROSTHESIS

(76) Inventor: Pierre Galvagni SILVEIRA, Florianopolis-SC (BR)

(21) Appl. No.: 13/447,842

(22) Filed: Apr. 16, 2012

Related U.S. Application Data
(63) Continuation-in-part of application No. 12/198,742, filed on Aug. 26, 2008, now abandoned.

(30) Foreign Application Priority Data
Nov. 30, 2007 (BR) PI 0704464-D

(51) Int. Cl. A61F 2/06

(52) U.S. Cl. 623/1.14; 623/1.13

(57) ABSTRACT

An endoprosthesis to be implanted in vessels damaged in a way as to be detrimental to the flow of the biological fluids they convey, intended for the treatment of aneurysms in areas of a vessel in the vicinity of a bifurcation, such as, for example, an aneurysm of the abdominal aorta.

### CROSS-REFERENCES AND RELATED APPLICATIONS

This application is a Continuation-in-part of U.S. Ser. No. 12/198,742, filed Aug. 26, 2008, the disclosure of which is expressly incorporated herein by reference in its entirety for any purpose.

### FIELD OF THE INVENTION

The present invention relates overall to a device and a method for repairing the arterial system at the point where a principal artery bifurcates into at least two secondary arteries.

### BACKGROUND

An aneurysm of the abdominal aorta is a dilatation of the walls of this vessel in the abdominal region. The aorta is the bodies' main artery, ascending from the heart's left ventricle to arch around and descend through the thorax and abdomen to finally divide into the two common iliac arteries that supply blood to the pelvis and lower limbs. Aneurysms usually occur in the abdominal part of the aorta below the kidneys. Failure to treat this condition may eventually result in the rupture of the dilatation (aneurysm) causing a massive haemorrhage in a very short period of time with fatal consequences. This is the reasons that treatments such as implanting a reinforcing prosthesis inside the dilated part of the aorta walls are vital to save patients lives. Despite abdominal aortic aneurysms being the most common, they are not restricted to the abdominal area. Aneurysms may also occur, for example, in the thoracic aorta.

The disruptions caused by of the abdominal aortic aneurysms are very serious and may lead to death. Until recently, treatment of aortic aneurysms consisted of invasive surgery methods for inserting a graft inside the aorta to reinforce the artery. Such a procedure requires a surgical incision to allow access to the vessel, which may result in rupture of the aneurysm due to the sudden reduction in the external pressure exerted by the neighboring organs and tissues, which are displaced during access procedure. Quite apart from this serious issue, other risk factors include loss of blood and consequent weakness, anemia and low blood pressure associated to the abdominal aortic aneurysm. As a result of the inherent risks and complexity of surgical procedures, several alternative devices and methods have been proposed for implanting a graft inside vessels for the treatment of aneurysms.

However, despite the advances represented by the use of stent and stent-graft devices, they have revealed failings both with regard their implanting processes and performance. As described in US 2006/184229, these failings may be classified into four main categories. Type I failings are related to the occurrence of leakage between the vascular endoprosthesis and the vessel walls in the area of the proximal aorta immediately above the aneurysm and, therefore, results in continued blood flow to the aneurysmal sac, which thus maintains the pressure at this point and favours continued expansion and consequent rupture of the aneurysm. Type I failings may also be caused by the irregular shape of the vessel and/or calcified topography of the aorta lumen which results in poorly inserted circular prostheses in non-circular lumens of the aorta. Type II failings are related to blood flowing through collateral vessels in the dilated area of the aneurysm which requires a further embolisation procedure. Type III failings are of mechanical origin and result from excessive wear of the metal/non-metal interface or the poor integrity of a connection or connections between the modular components of a prosthesis. Lastly, the type IV failings are related to excessive porosity of the prosthesis walls, which allows the blood to migrate through the walls despite the soundness of all mechanical seals and connections. To remedy usual Type I failings, US 2006/184229 proposes an implantable prosthesis with a radially expandible tubular body with at least one flap extending through it.

Vorwerk in U.S. Pat. No. 5,562,724 describes an endovascular graft prosthesis to be positioned in (or close to) a bifurcation of the arterial system of a patient, with this prosthesis comprising a main tubular body having a bag-shape and provided with two outlet openings wherein the said main body is intended for location in the principal upstream artery above the bifurcation and having tubular legs joining the main body and adapted to extend into the two downstream arteries. The positioning of the main body in relation to the radially expansive stent devices and the entire assembly in the arteries is done through the use of guide wires. Although such a device does represent an advance compared to former techniques involving surgical procedures, it still presents failings related to its unfavourable displacement along the blood flow and the precision required when correctly positioning the device at the time of implant. The device described in US 2007/027531 also uses a system of guide wires to facilitate the implant operation of the device, which comprises at least one filamentous tubular member having a distal extremity and a proximal extremity with a hollow nucleus to receive the guide wire that helps position the device at the intended location.

U.S. Pat. No. 6,802,859 to Pazienza proposes a bifurcated graft implant having a trunk portion and a portion with independent flexible legs wherein the entire assembly (main portion+legs) may constitute a unitary body or be formed of modular elements. To ensure the flexibility of the bifurcation, this graft is supported by a stent lattice throughout. Despite this device being self-expanding and having appropriate flexibility at the region of bifurcation, it remains difficult to implant at the intended location.

Another device for the repair of abdominal aortic aneurysms is proposed in U.S. Pat. No. 6,942,691 to Chuter. This device comprises a modular graft that includes two elements configured to be inserted into each other over an extension sufficiently long as to form a resistant seal yet remaining flexible enough to adapt to the region of the bifurcation. This device comprises a first and second modular element with each expanding from an originally compressed state so as to allow implantation at the intended aneurysm location. The graft described by Chuter is practical since it allows insertion of both component elements at an intended location but, nevertheless, it presents inconveniences related to the stability of its placement and the relative safety of the large blood flow expected through it.

A solution intended to facilitate the implant of a device in the region of an aneurysm of the abdominal aorta is proposed in document US 2003/120338 of Chobotov et al. This solution relates to providing means to allow the use of a catheter having a very small diameter in the delivery systems for devices within the bodies of patients. The proposed device includes a graft having proximal and distal extremities and is provided with a connector member arranged or fixed at one or both extremities, having one or more connector elements wherein the said connector member may be enclosed within multiple layers of the graft body section. Despite this solution being of interest due to the use of a reduced diameter catheter, this device described in US 2003/120338 is complex and presents the disadvantage of being difficult to position correctly at its intended location. A similar device also presenting the same disadvantage is described in document US 2006/173533 (corresponding to EP 1464301).

To correct the issues of stent graft instability, unwanted displacement from the required position and material fatigue, document WO 2001/67993 of Cook Inc. proposes a stent graft assembly comprising a main body having an ipsilateral leg and a contralateral stump that, combined, form a bifurcation at the distal extremity. A delivery system for this stent graft assembly is also proposed.

Document WO 2006/014952 (corresponding to US 2006/025850 of Frederik et al.) describes an endoprosthesis comprising (i) a main body having a tubular structure configured to attach firmly to a vessel and serve as a seal preventing blood from reaching the aneurysm, (ii) a section constituted of two legs allowing the passage of fluids to the main body and having multiple stent elements and, (iii) a graft attached to the main body and the two legs. This type of endoprosthesis presents the disadvantage of greater implanting difficulty since it consists of a unitary body insufficiently flexible to adjust well at its intended position.

Another fundamental aspect for the treatment of aneurysms using endoprosthesis relates to the methods of implanting these and appropriate means for this delicate operation.

An endoprosthesis delivery system for the adequate implant of stents or graft stents should incorporate the following desirable features: (a) A minimum diameter should be maintained during insertion to avoid trauma and/or unneccessary difficulties during the implanting operation; (b) Means to facilitate the precise visualisation and positioning of the delivery catheter to ensure correct positioning of the device; (c) Reliable and reproducible expansion of the metal structure and graft metal structure allowing complete expansion to operational diameter with no deviations to the degree or efficiency of expansion; (d) Reliable and reproducible uncoupling or release of the metal structure and the graft metal structure from the catheter body; (e) Efficient retrieval or removal of the delivery catheter without disturbing the recently implanted stent or graft stent, and (f) Easy of verifying the occurrence of biological fluid (i.e. blood) leakage around the endoprosthesis (endo-leakage) following delivery and implantation within the vessel.

Several delivery systems have been proposed aiming to ensure these features. For example, U.S. Pat. No. 6,379,372 to Dehdashtian et al. (corresponding to PI 9712034) describes a delivery and implant system for use inside a body lumen (e.g. a blood vessel) for a radially expandable endoluminal prosthesis with the system comprising: (a) a delivery catheter; (b) an introducer assembly, and (c) a dilator. Despite this system allowing the safe introduction of an expandible endoluminal prosthesis, it presents major limitations, such as those relating to providing the means for performing the expansion of the prosthesis in an aneurysm in the vicinity of a bifurcation (i.e. an abdominal aortic aneurysm) as well as preventing the control of fine adjustments required to any of the endoprosthesis components once implanted at the intended location. The delivery devices described in documents U.S. Pat. No. 6,673,102 to Vonesh et al. (corresponding to CA 2,503,480) and U.S. Pat. No. 6,872,224 to Teixeira Moreira et al. (corresponding to PI 9900959) allow greater flexibility for adjusting the different portions of the endoprosthesis at their place of implant and use small diameter catheters but, nevertheless, present the same limitations as the system described in U.S. Pat. No. 6,379,372.

Document U.S. Pat. No. 7,112,217 to Kugler et al. describes a delivery system and method for an endoprosthesis that allows adjustment of the various parts at the place of implant. However, this system and method presents the disadvantage that the link between the main body and the legs of the endoprosthesis is based on the coupling of stents fitted to the extremities of these parts and, furthermore, requires an incision of the artery to introduce the endoprosthesis implanting catheter.

Document US 2001/037142 of Stelter et al. reveals a delivery system and method for endovascular devices comprising: (i) a first sheath with distal and proximal extremities and at least a first expandable device at the proximal extremity; (ii) a second movable sheath inside the first sheath having respective distal and proximal extremities and containing a second expandable device, and (iii) trigger buttons linked to the first and second expandable devices. Despite this system allowing the implant of an endoprosthesis and the adjustment of the various component parts, it neither provides the means of fine adjusting nor correcting the position of the endoprosthesis during the implant operation. The endoprosthesis delivery systems described in WO 01566504 (corresponding to documents US 2006/224227 and US 2003/220681) also present the same limitations.

Document US 2006/036314 describes a delivery system for endoprosthesis that allows implanting the device in a bifurcated vessel but, however, this system does not allow any means of fine adjusting or correcting the position of the endoprosthesis during the implant operation.

Document US 2006/085012 of Dolan illustrates a procedure for implanting an endoprosthesis using a delivery system without, however, describing implanting in a bifurcated vessel which is an operation requiring further steps for expanding the different parts of the endoprosthesis, such as, for example, the main body and the legs extending into the arteries branching from the trunk vessel in which the main body of the endoprosthesis is located. The delivery system described in document US 2006/142836 of Hartley et al. also presents similar failings. However, the delivery system described in US 2006/276872 of Arbefeuille et al. (corresponding to PI 0414109) is intended for implanting this type of device in a curved vessel (i.e. the arched part of the aorta) where guide wire type delivery systems such as those described in documents WO 02051336 of Bourne et al. and WO 2005/039442 of Boston Scientific cannot be used. Despite the system described in US 2006/276872 being appropriate for curved sections of vessels such as the aorta, it does not meet the requirements for implanting endoprosthesis in the vicinity of bifurcations and neither provides a means for correcting the position of the endoprosthesis during the implant process.

Frid in U.S. Pat. No. 6,149,682 teaches a modular endoprosthesis with symmetrical configuration and with multifilament. Frid further teaches the construction with the same mandrel to produce one body, the body is then divided in two after a heat treatment. In Frid there are interconnections that give a long deformation for the bodies creating some problems with the sutures between structure and graft. However, this endoprosthesis will likely have problems with vascular fixation (low radial force) or with introduction of the endoprosthesis in the delivery system, due to excessive material to be insert in a 16Fr profile, as a reduction of metal quantity would enables the assembly of the endoprosthesis in a percutaneous delivery system (16Fr).

Another important drawback in Frid is the construction with the same mandrel to produce one body and then divided in two after heat treatment. In Frid there are these interconnections that give a long deformation for the bodies creating some problems with the sutures between structure and graft.

Although Frid also discloses a structure comprised of two bodies, the structure cannot be stitched to the graft in at least two points. Also the two bodies cannot be coupled in a manner to form a sealed connection impeding leakage of the blood from inside the endoprosthesis to the aneurysm.

Iancea in US Publication 2003/0176911 teaches a modular endovascular graft device for treating vasculature, wherein the graft is fixed in the structure with sutures. Components in Iancea are reinforced with a thin coating of a biocompatible elastomer applied to the graft material, such as a polyurethane co-polymer dip-coated applied onto the surface of the graft material. An important drawback of Iancea is that the graft, polymeric sutured film/fabric of or the elastomeric grafts applied by dipping process cannot be stretched without damage.

Also, In Iancea, the first and second graft components are mechanically interconnected. The first graft component has a radially adjustable structure having a thread configured into a form of a lasso further comprising a plurality of slip knots.

Another drawback found in Iancea is that first graft component needs a radially adjustable structure together with a second graft component suitable so that when placed in contact with the first component creates a radially adjustable structure; what is detrimental if compared to other devices or structures that do not need to be geometrically compatibles, such as of the present application.

All these features in Iancea, discussed above, are existent in the present invention and would prevent the technique of the modular endovascular graft device of Iancea to be used with the modular endoprosthesis of Frid. It is important to understand and reinforce that the graft, polymeric sutured film/fabric taught by Inacea or elastomeric grafts applied by dipping process also taught by Iancea on multifilament stents taught by Frid, cannot be stretched without damage.

Although the endoprosthesis and implanting systems mentioned above represent significant advances, mainly since they replace surgical techniques, it remains necessary to improve these devices as well as the methods for implanting them and applying them to varying biological conditions. Therefore, the present invention is directed at providing a safe and efficient means to treat aneurysms, including abdominal aortic aneurysms and thoracic aortic aneurysms thus eliminating a series of difficulties associated with current endoprostheses and their delivery systems. One of the aims of the endoprosthesis of the present invention is the prevention of endo-leakage disrupting the normal dynamic of vascular fluids by providing a device that is simple to position and replace, is an efficient fluid seal and, furthermore, has a configuration that avoids any displacement at the place of implant without, however, interfering with the normal blood flow either in the actual vessel of the aneurysm or those vessels deriving from it. In summary, the device disclosed herein has the capacity of remaining firmly fixed and sealed tight when placed in bifurcated, tortuous, sharply curved or oddly shaped vessels, partially damaged or calcified vessels and either short or long vessels. Furthermore, the device of the present invention is extremely durable, extensible and may be reconfigured but, nevertheless, remains in position at the place of implant and maintains the sealing capacity required of an endoprosthesis.

### SUMMARY OF THE INVENTION

The invention intends to provide an endoprosthesis to be implanted in vessels damaged in a way as to be detrimental to the flow of the biological fluids they convey. More especially, the invention is intended for the treatment of aneurysms in areas of a vessel in the vicinity of a bifurcation, such as, for example, an aneurysm of the abdominal aorta.

More specifically, the invention is intended for the repair of an aneurysm in the vicinity of the aorta. The invention relates to a modular intravascular device termed a self-expanding percutaneous endoprosthesis used in the treatment of aneurysms, more specifically of the abdominal aorta. The endoprosthesis constructed with a monofilament of NiTi wire with a PET fabric graft and is composed of two complementary bodies, namely, body “A” and body “B”, which both comprise self-expanding metal structures (a set of several stents) and a graft of appropriate tissue. The self-expanding construction is a characteristic of NiTi material. The endoprosthesis is suitable to be implanted in a patient needing such treatment.

The present invention seeks to provide a modular endoprosthesis comprising: (i) a body A and a body B, constructed with a self-expanding monofilament of NiTi wire with a PET fabric or ePTFE graft; the bodies being formed by proximal, intermediate and distal regions; (ii) the said bodies A and B have a portion of greater diameter that divides, in a bifurcating portion, in at least two legs with one being significantly shorter than the other; (iii) the said proximal region of said body A comprises a structure of resistant and flexible material wherein a free end comprises a stent without graft having a configuration appropriate for attachment to the vessel wherein the remainder of the said body A comprising the said metal structure is stitched to the graft in at least two points; (iv) the said intermediate region of the said bodies A and B comprises a structure of resistant and flexible material stitched to the graft; (v) the said distal region of the said bodies A and B have laser cuts Z stents without graft comprising a configuration appropriate for attachment to the vessel walls, and (vi) the said bodies A and B are coupled in a manner that the said body B remains positioned within the said body A forming a sealed connection impeding leakage of the blood from inside the endoprosthesis to the aneurysm and the disconnection of the modules bodies A and B.

### BRIEF DESCRIPTION OF THE DRAWINGS

FIG. 1 is an illustration of body “A” of the invention endoprosthesis.

FIG. 2 is an illustration of body “B” of the invention endoprosthesis.

FIG. 3 is an illustration of bodies “A” and “B” assembled, forming the invention endoprosthesis.

FIGS. 4 and 5 are illustrations of proximal stents with the radiopaque indicator.

FIG. 6 is an illustration of stents with the holes for the insertion of the radiopaque indicators/markers.

FIG. 7 is an illustration of the orientation of bodies A and B done through the radiopaque marks/indicators in the shape of a letter “a”.

### DETAILED DESCRIPTION OF THE INVENTION

The endoprosthesis of the present invention is intended for the treatment of anomalies of the vessels conveying biological fluids (i.e. blood), more specifically in humans. More specifically, the invention endoprosthesis is percutaneous, which thus reduces or eliminates the surgical procedure normally necessary in cases of vessel deformation, such as, for example, blood vessels, with the said percutaneous endoprosthesis being preferably used in the treatment of aneurysms, preferably of aneurysms occurring in the vicinity of bifurcations of the main vessel, more preferably of aortic aneurysms and, yet more preferably, abdominal aortic aneurysms. The fact that the device of this application is percutaneous allows it to be implanted in the patient by mere puncture and, therefore, without needing to dissect any vessels (e.g. the iliac arteries) to allow access of the delivery system consisting of catheters that is required to position and release the implant (endoprosthesis). Without doubt, this constitutes a less aggressive system for the patient, avoiding the risks inherent to conventional treatment and thus reducing recovery time.

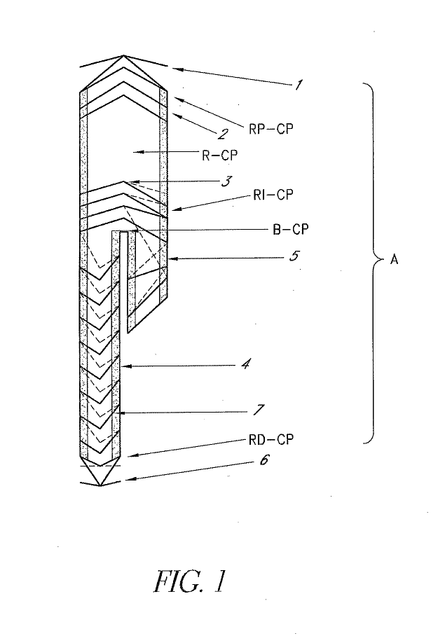

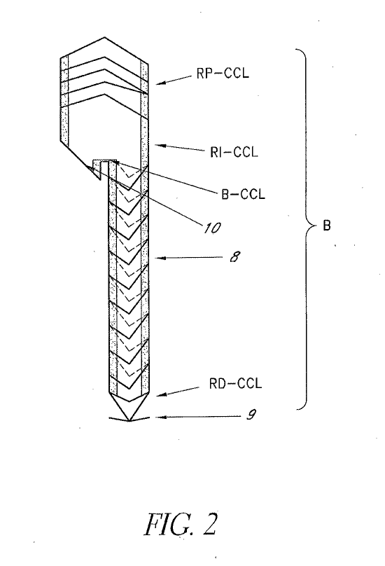

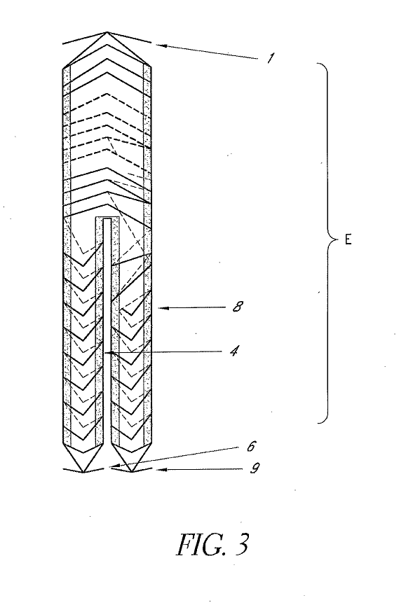

As it can be seen in FIG. 3, the endoprosthesis (E) of the present invention comprises a body A and a body B, wherein both these bodies are composed of a laser cut stable yet flexible metal structure, preferably a metal structure formed by a series of self-expanding stents and by the graft R-CP (FIG. 1) and R-CCL (FIG. 2), that cover most of the structure of stable material. Body A and body B are divided into three regions, namely; a proximal region, an intermediate region and a distal region.

The terms “proximal” and “distal” as used herein are intended to mean the portion closer to the heart and the portion furthest from the heart, respectively.

The proximal region of body A (RP-CP) presents a stent without a graft at its upper extremity (proximal) (1) or, therefore, graft free. This free stent is constructed with nitinol wire, which is a nickel-titanium alloy, having the main purpose of fixing the device to the walls of a vessel such as the aorta. As can be seen in FIG. 1, the free stent (1) has the shape of a quadruple Z stent laser cut. In the case that the said quadruple Z stent is formed by laser cut NiTi tube; and may be fitted with additional means of attachment, such as barbs or hooks that help fix the free stent (1) to the artery walls. Preferably, the free stent (1) is encapsulated partly in the coating material, which contributes to reducing the transverse section of the endoprosthesis material (E).

It is important to note that the shape of the quadruple Z of the free stent (1) confers the endoprosthesis good resistance to possible displacement induced by the flow of biological fluids, i.e. blood, in the vessel. It should also be noted that endoprosthesis migration is a frequent and recurrent problem occurring with the devices presently available on the market.

With the intent of perfecting the attachment and adjustment of the endoprosthesis to the artery shape, the proximal region of the main body (RP-CP, in FIG. 1) can also be fitted with two or more saddle shaped wires stitched to the graft (R-CP, in FIG. 1) thus forming rings (2) that conform to the artery shape and make the endoprosthesis occupy the entire perimeter of the artery. Therefore, these rings (2) serve the purpose of seals for the proximal region and thus prevent blood flow from inside the endoprosthesis (E) to the artery walls. It should be noted that, preferably, a part of the main body does not have the metal structure allowing good adjustment of the endoprosthesis (E) in tortuous arteries.

It is also possible to note in FIG. 1 that the intermediate region of body A (RI-CP), presents a structure comprising a single shaped wire (3). This wire (3) has the purpose of maintaining the graft open and thus allowing unobstructed blood flow through the vessel. Optionally, this structure may also be reduced to a minimum leaving the contralateral body B to keep the endoprosthesis (E) open. The said intermediate region of the main body (RI-CP) contains a bifurcation (B-CP) from which two legs extend. One of these legs, leg (4), is longer and shall be positioned within one of the main artery branches such as the iliac artery at the time of implant. The shorter leg, leg (5), has the purpose of serving as a seal between body A and body B.

FIG. 1 further shows that the distal region of body A (RD-CP) also has a free stent (6) with the purpose of fixing the distal extremity of the endoprosthesis (E) to the walls of the vessel. Preferably, this free stent (6) is similar to free stent (1) having the shape of a quadruple Z stent partially coated. Optionally, this region may be fitted with additional means of attachment, such as barbs or hooks that help fix the device. The metal structure (7) of leg (4) is produced with NiTi wire and laser cut stent in the distal regions, they have helicoidal shape and Z stent shape, respectively. The leg (4) is produced with the same material used in the intermediate region of body A (RI-CP).

The materials used for the graft are natural, artificial or synthetic fibrous materials, coated or not, known in the art. The graft described in documents WO 2002/15951, PI 9608191 (corresponding to applications WO 9633066 and US 2005/096737) and WO 2005/025456 (corresponding to document CA 2539110) may be cited as examples. Preferably, the graft material is made of polyester (polyethylene terephthalate) or expanded polytetrafluorethylene (PTFE).

FIG. 2 shows body B of the endoprosthesis of the present invention. Body B is shorter than body A, but also has a wire stent with helical shape and a laser cut stent in the distal regions metal structure, throughout its entire available transverse section that maintains the graft open. Optionally, the proximal region of body B (RP-CCL) may be provided with a free stent that may be similar to free stent (1) of the main body, or, alternatively, may be a conventional Z-stent (a Z shaped wire). The free stent of body B may be designed to occupy the portion of the main body without a metal structure thus increasing the rigidity and stability of the device when assembled.

The intermediate region of body B is provided with a bifurcation from which a contra-lateral leg extends, leg (8), that shall be positioned inside another branch of the main vessel (e.g. the iliac artery) when implanting the endoprosthesis (E). An opening (10) allowing blood flow is located to the side of the contra-lateral leg (8).

In this manner, both body A and body B are complementary and form a perfect bifurcation (E) without any possibility of becoming disconnected. The walls of the main body are formed from a double layer of graft, which twice thus confers twice the protection against the effects of the repeated secondary demands imposed by the pulsing of biological fluids (e.g. blood).

The distal region of body B (RD-CCL) is provided with a free stent (9) that, preferably, is similar to free stent (6) of the leg (4) of the main body (body A), or, alternatively, may be a Z-stent.

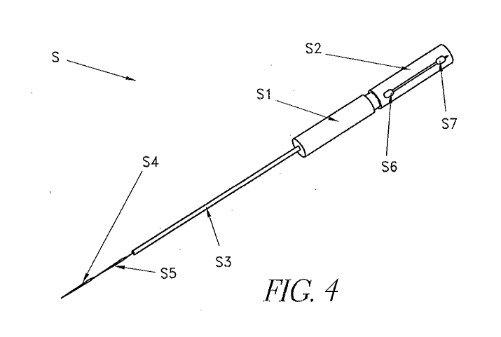

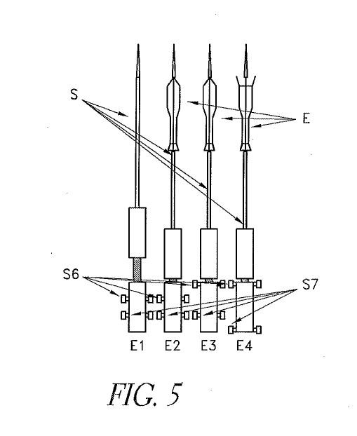

As it can be seen in FIGS. 4 and 5, laser cut proximal stent (1) and distal stents (6) and (9) have radiopaque indicators (12), to guide the longitudinal positioning and placement of bodies A and B.

FIG. 3 shows the endoprosthesis (E) of the present application entirely assembled. Bodies A and B are assembled by inserting body B inside body A. The body B is inserted into body A thought the opening (5). The opening (10) of body B is aligned with the longer leg (4) of body A and the longer leg (8) of body B passes through the short leg (5) of body A. This configuration allows free blood flow to both branches of the trunk vessel (e.g. the two iliac arteries). Furthermore, the superposition of the short leg (5) of body A and the long leg (8) of body B forms a seal between both bodies and prevents blood leakage.

Different from other devices currently available on the market that are based on the concept of two superposed endoprosthesis (a bifurcated main body and a contra-lateral extension), the configuration of the endoprosthesis of the present invention confers the device good stability and completely eliminates any possibility of contra-lateral leg disconnection. Bodies A and B of the present application are not symmetrical and may be produced with different mandrels. Also, the reduction of metal quantity (bodies A and B not symmetrical) enables the assembly of the endoprosthesis in a percutaneous delivery system. The radial force of braided stents is known to be lower than laser cut stents.

The division of the device structure into two bodies (A and B) constitutes a further advantage of the endoprosthesis of the present invention because it divides the material area and thus makes it possible to implant both bodies separately using small calibre catheters (e.g. a 14F catheter). Furthermore, the endoprosthesis of the present invention may be provided with sensory means for measuring and monitoring the patient's condition and position, such as, for example, the sensor device described in document WO 2004/105637.

The endoprosthesis is constructed with a monofilament of NiTi wire with a PET fabric graft and body A and body B, both comprise self-expanding metal structures (a set of several stents) and a graft of appropriate tissue. The self-expanding construction is a characteristic of NiTi material.

The deformations of the braided NiTi stent will be so long that any graft or suture will support without damage.

Bodies A and B may be constructed as an aortobi-iliac body (bifurcated body), or as well as an aorto-uni-iliac body. The aorto-bi-iliac construction is obtained through hemodynamic intervention performed through iliac or femoral vessels accessed by right and left. The aorto-uni-iliac construction is done by both bodies A and B through one unique iliac or femoral artery. This procedure is necessary when it is not possible insert the delivery system through one of the sides from iliac or femoral artery but at the same time has no indication to traditional surgery.

The orientation of bodies A and B is done through the radiopaque marks/indicators in the shape of a letter “a” (13). The frontal, lateral and posterior visualizations are easily identified by fluoroscopy while the endoprosthesis is still inside the delivery system. The visualization of the “a-shape”, as seen in FIGS. 1, 2, 3, 5 and 7, indicates that the endoprosthesis is in the frontal orientation, and so, in the correct orientation. Both bodies A and B have such identification and the superposition, or overlaping, of both radiopaque markers in the “a-shape” during the implanting, indicate the correct longitudinal positioning This indication is a correct indication for both, the aorto-bi-iliac body and aorto-uni-iliac implants.

When bodies A and B in the present application are positioned to forming one bifurcated body, body B needs a specific shape not to close the long leg of body A and to maintain the patency of both legs, avoiding any unwanted obstruction. As it can be seen from the shapes of the bodies, the lengths of the bodies A and B are different, and this difference is needed to reduce the risks of renal artery obstructions in the proximal portion of the assembled parts.

All patent applications and publications mentioned in the above description are indicative of the level of expertise of those skilled in the art relating to the invention. All the patent applications and publications are included herein as reference in the same extent that each individual patent application or publication was specifically indicated to be indicated as reference.

Despite the above invention having had certain details described by means of illustrations and examples for the purpose of greater clarity and understanding, it remains obvious that certain alterations and modifications may be undertaken within the scope of the claims that accompany this description.

1. A modular endoprosthesis comprising:
(i) a body A and a body B, constructed with a self-expanding monofilament of NiTi wire with a PET fabric or ePTFE graft; the bodies being formed by proximal, intermediate and distal regions;
(ii) the said bodies A and B have a portion of greater diameter that divides, in a bifurcating portion, in at least two legs with one being significantly shorter than the other;
(iii) the said proximal region of said body A comprises a structure of resistant and flexible material wherein a free end comprises a stent without graft having a configuration appropriate for attachment to the vessel wherein the remainder of the said body A comprising the said metal structure is stitched to the graft in at least two points;
(iv) the said intermediate region of the said bodies A and B comprises a structure of resistant and flexible material stitched to the graft;
(v) the said distal region of the said bodies A and B have laser cuts Z stents without graft comprising a configuration appropriate for attachment to the vessel walls, and
(vi) the said bodies A and B are coupled in a manner that the said body B remains positioned within the said body A forming a sealed connection impeding leakage of the blood from inside the endoprosthesis to the aneurysm and the disconnection of the modules bodies A and B.

2. The modular endoprosthesis according to claim 1, wherein the endoprosthesis is a percutaneous endoprosthesis.

3. The modular endoprosthesis according to claim 1, wherein bodies A and B are constructed as an aorto-bi-iliac body or as an aorto-uni-iliac body; wherein the aorto-bi-iliac construction is obtained through hemodynamic intervention performed through iliac or femoral vessels in the both sides (right and left); and wherein the aorto-uni-iliac construction is obtained by both bodies A and B through one unique iliac or femoral artery.

4. The modular endoprosthesis according to claim 1, wherein the proximal stent and distal stents have multiple holes allowing for the insertion of radiopaque indicators; the radiopaque indicators orienting the longitudinal positioning and placement of bodies A and B.

5. The modular endoprosthesis according to claim 4, wherein the orientation of bodies A and B is done through the radiopaque indicators in the shape of a letter “a”, wherein the frontal, lateral and posterior visualizations are identified by fluoroscopy while the endoprothesis is still inside a delivery system, and wherein a superpositioning of both radiopaque markers with “a-shape” during the implanting, indicate the correct longitudinal positioning

6. The modular endoprosthesis according to claim 5, wherein the visualization of the “a-shape” indicates that the endoprothesis is in a frontal orientation, and so, correct orientation.

7. The modular endoprosthesis according to claim 1, wherein the shape of the free extremity without graft of the proximal and distal region of said bodies A and B are, respectively, quadruple Z stent and Z stent laser cut.
